(19) United States
(12) Patent Application Publication
(10) Pub. No.: US 2019/0380482 A1
(43) Pub. Date: Dec. 19, 2019

(54) ORAL CARE DEVICE AND ATTACHMENT

(71) Applicant: KONINKLIJKE PHILIPS N.V., EINDHOVEN (NL)

(72) Inventors: MARTIN JOHN EDWARDS, SOLIHULL (GB); JAYPRAKASH PATEL, WEMBLEY (GB)

(21) Appl. No.: 16/465,611

(22) PCT Filed: Nov. 22, 2017
(86) PCT No.: PCT/EP2017/079981

§ 371 Date: May 31, 2019

Related U.S. Application Data
(60) Provisional application No. 62/428,765, filed on Dec. 1, 2016.

(51) Int. Cl. A46B 15/00; A46B 9/04; A61N 1/05; A61C 17/22

(52) U.S. Cl. CPC A46B 15/0022; A46B 15/0081; A46B 2200/1066; A61N 1/0548; A61C 17/22; A46B 9/04

(57) ABSTRACT

There is provided an attachment for an oral care device, and an oral care device comprising such an attachment. The attachment comprises a treatment element. The treatment element includes a first electrode and a second electrode, and the electrodes are arranged such that, when the first and second electrodes are placed in contact with tissue within an oral cavity of a user, an applied current is able to flow from the first electrode to the second electrode, via the tissue.

### TECHNICAL FIELD OF THE INVENTION

The invention relates to an oral care device and an attachment for such a device. More particularly, the invention relates to and attachment having electrodes.

### BACKGROUND TO THE INVENTION

A dry mouth (known as xerostomia) can occur when salivary glands in the mouth do not produce enough saliva. Such a condition may be caused by, for example, dehydration or anxiety. A dry mouth, and an inability of salivary glands to produce enough saliva can lead to symptoms which can be unpleasant for an affected person. Such symptoms can include a burning sensation or soreness in the mouth, dry lips, bad breath (known as halitosis), a decreased or altered sense of taste, oral infections, tooth decay, gum disease and a reduced ability to speak, eat and/or swallow.

Short-term treatments of xerostomia include such actions as chewing gum, or sucking mints. However, such treatments can have other undesired effects on tissue within the oral cavity, such as demineralization of teeth, or irritation of the oral mucosa, and have only short-term benefits.

To reduce symptoms resulting from xerostomia, it is desirable that salivary glands within the mouth produce sufficient saliva. Existing means for promoting saliva generation can be ineffective. Therefore, there is a need for a more effective means for improving saliva production within an oral cavity.

### SUMMARY OF THE INVENTION

According to a first aspect, there is provided an attachment for a personal oral care device. The attachment includes a first electrode and a second electrode, the electrodes arranged such that, when the first and second electrodes are placed in contact with tissue within an oral cavity of a user, an applied current is able to flow from the first electrode to the second electrode, via the tissue.

Applying electrical currents to oral tissue can help to stimulate saliva production by salivary glands. Specifically, electrical stimulation of particular nerves in the oral cavity can stimulate saliva production. A tongue includes a high density of nerve connections. By incorporating electrodes into an oral care device, such as a tongue cleaner or a power toothbrush, a user is able to apply electrostimulation signals and perform their regular oral hygiene routines conveniently, using the same device. Including the electrodes in the attachment in such a way that both electrodes are arranged to contact the oral tissue to be treated is advantageous in that particular portions of the oral tissue can be targeted. A controlled current may be delivered to a particular portion of the tissue intended to be treated, without inadvertently delivering a current to other portions of the oral cavity.

Furthermore, the action (e.g. vibration or rotation) of the oral care device attachment on the oral tissue may serve to assist or improve the effect of the electrostimulation by breaking down, penetrating and/or dispersing material (e.g. a biofilm or a saliva film) on the tissue so that the electrodes can contact the tissue more cleanly.

The first electrode and the second electrode may be of opposite polarities. By positioning an anode and a cathode on a particular portion of tissue in a user's mouth, the current flow through the tissue may be accurately controlled and targeted as desired.

In some embodiments, the first electrode and the second electrode may be at least partially embedded within, located on, or extending from a surface of the attachment.

In some embodiments, the oral care device attachment may comprise a tongue cleaning attachment.

The tongue cleaning attachment may comprise a plurality of cleaning elements. The first electrode may comprise a first subset of the plurality of cleaning elements. The second electrode may comprise a second subset of the plurality of cleaning elements. The cleaning elements may be cone-shaped protrusions, ridges or other types of protrusions arranged to complement, or fit into recesses and troughs in a surface of a tongue, in order to improve the cleaning effect. By forming the electrodes in a shape similar to the shape of the cleaning elements, the contact between the electrodes and the oral tissue may be improved, thereby improving the electrostimulation effect.

At least one of the first electrode and the second electrode may comprise at least one of a ridged surface and a surface having a plurality of protrusions.

In some embodiments, the oral care device attachment may comprise a toothbrush head.

The attachment may comprise a first surface and a second surface opposite to the first surface. A first plurality of bristles may extend from the first surface and the first electrode and the second electrode may be located on or in the second surface. In this way, a user may use the device for brushing his or her teeth, and for providing an electrostimulation treatment separately, using the same device. The application of the electrostimulation signals may be performed without disrupting the user's regular oral hygiene routine.

In some embodiments, the toothbrush head may comprise a plurality of bristles. The first electrode may comprise a first subset of the plurality of bristles. The second electrode may comprise a second subset of the plurality of bristles. By forming the electrodes from some of the bristles of the toothbrush, a user may apply the electrostimulation treatment while brushing his or her teeth, thereby reducing the time required to perform both tasks.

Each of the bristles in the first subset and the second subset may comprise an insulated portion along a portion of its length. Insulating a portion of each of the bristles forming the electrodes reduces the chance of current flowing between the electrodes via a medium (e.g. saliva or toothpaste) other than the intended tissue.

In some embodiments, the first and second electrodes may be formed at least partially of a material selected from a group comprising: gold, and an electrically conducting polymer. The first and second electrodes may be formed from other metals, and/or other biocompatible materials.

According to a second aspect, there is provided an oral care device comprising a handle having a body; a drive mechanism within the body of the handle; a drive shaft configured to be driven by the drive mechanism; an attachment as described above; and a signal generator to generate a first electrical current for delivery to at least one of the first electrode and the second electrode. The signal generator may be configured and/or controlled (for example by a processor) to generate a desired current and/or voltage waveform.

The signal generator may, in some embodiments, be located within the attachment. In other embodiments, the signal generator may be located within the body of the handle.

In some embodiments, the attachment may comprise a third electrode and a fourth electrode. The signal generator may be configured to generate a second electrical current for delivery to at least one of the third electrode and the fourth electrode, the second electrical current being different from the first electrical current. In other embodiments, the current delivered to the third and/or fourth electrodes may be the same as the current delivered to the first and/or second electrodes. By including additional electrodes in the attachment, electrostimulation treatment may be targeted to specific portions of tissue, and a specific current may be applied to each electrode or pair of electrodes, as desired.

The signal generator may be configured to generate electrical current in a series of pulses.

Other advantageous features will become apparent from the following description.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a schematic drawing of an oral care device;

FIGS. 2a and 2b are schematic drawings of an attachment for an oral care device;

FIGS. 3a and 3b are schematic drawings of an example of a tongue cleaning attachment for an oral care device;

FIGS. 4a and 4b are schematic drawings of a further example of a tongue cleaning attachment for an oral care device;

FIGS. 5a and 5b are schematic drawings of an example of a toothbrush attachment for an oral care device;

FIGS. 6a and 6b are schematic drawings of a further example of a toothbrush attachment for an oral care device; and

FIGS. 7a and 7b are schematic drawings of a further example of a toothbrush attachment for an oral care device.

### DETAILED DESCRIPTION OF THE PREFERRED EMBODIMENTS

As noted above, the present invention provides a device for promoting saliva production in an oral cavity. In particular, it has been recognised that electrically stimulating tissue within the oral cavity, such as the tongue, cheeks or gums, can help to improve saliva production by the salivary glands. More specifically, electrical stimulation of the oral mucosa—a mucous membrane lining the inside of a person's mouth—can lead to long-term improvement in saliva production, and can help to alleviate symptoms associated with xerostomia.

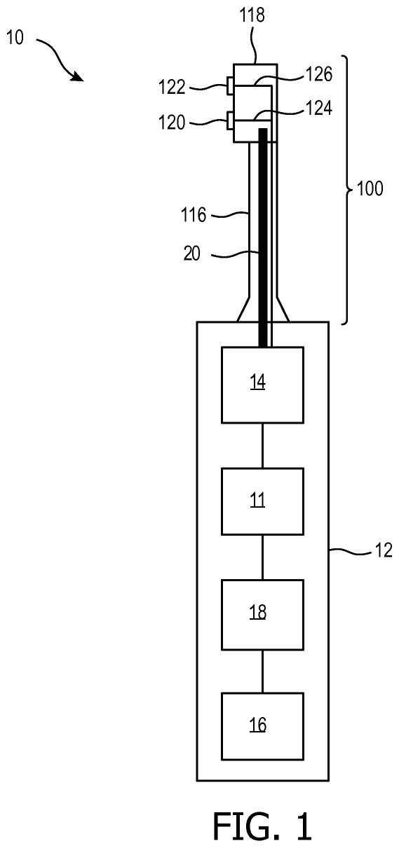

FIG. 1 is a schematic drawing of an example oral care device 10. The device 10 includes a body 12 and an attachment 100. The body 12 forms a handle of the device 10 which is shaped and sized to be held in a hand of a user during use. In some embodiments, the body/handle 12 may be substantially cylindrical in shape. The cavity within the body 12 houses various components of the device 10, which may include a controller 16, a battery 18, a signal generator 11 and a drive mechanism 14. The drive mechanism may drive a drive shaft 20 which may cause at least a portion of the attachment 100 to undergo some motion, for example vibration or rotation.

The function of the attachment 100 may be different in different embodiments, as discussed below. In general, however, the attachment 100 includes a treatment element 118, and may include a neck portion 116. The neck portion 116 of the attachment 100 may be arranged to couple to the body 12. However, it will be understood that, in some embodiments, the treatment element 118 may couple to the body 12 directly, without the use of the neck portion 116. In some embodiments, the neck portion 116 includes a cavity configured to receive the drive shaft 20 so as to secure the attachment 100 to the body 12, and to provide a connection between the drive shaft and the treatment element 118 of the attachment.

The treatment element 118 of the attachment 100 includes a first electrode 120 and a second electrode 122, which may be connected to one or more of the controller 16, the battery 18 and the signal generator 11, for example by connectors 124 and 126 respectively. When the oral care device 10 is switched on, for example by a user pressing a power button (not shown), the controller 16 may operate the battery 18 to supply power to the drive mechanism 14. The drive mechanism 14 may cause the drive shaft 20 to undergo movement, such as a vibration or a rotation, which may cause the treatment element 118, or a portion thereof, to move in a similar way. In use, a rapid movement of the treatment element 118, such as a vibration or a rapid rotation against oral tissue may help to penetrate, dislodge and/or remove a biofilm or a film of saliva on the tissue to be treated. In addition to operating the drive mechanism 14, the controller 16 may also operate the signal generator 11 to generate a current to be delivered to one or both of the electrodes 120, 122.

In some embodiments, the first electrode 120 and the second electrode 122 are of opposite polarities. In other words, one of the electrodes functions as an anode while the other electrode functions as a cathode. In use, a user of the device 100 inserts the treatment element 118 into his or her mouth and touches the first and second electrodes 120, 122 against oral tissue, such as their tongue. With the device 10 switched on, a current is caused to flow from one of the electrodes 120, 122 via the oral tissue, to the other of the electrodes. As the current flows through the oral tissue, the tissue may be stimulated in a process known as electrostimulation, which may cause stimulation of neural pathways of salivary glands, such as the submandibular gland and the sublingual gland, thereby inducing saliva generation.

The form of the current generated by the signal generator 11 may vary depending on the requirements of the device 10 or the desired outcome from using the device. For example, in some embodiments, the controller 16 may operate the signal generator 11 to generate a continuous current (e.g. a direct current) to flow via oral tissue between the first electrode 120 and the second electrode 122. In other embodiments, the controller 16 may cause the signal generator 11 to generate current in a pulsed form.

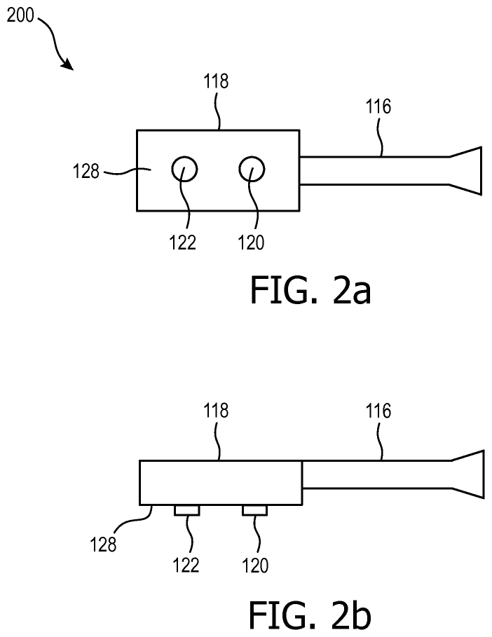

FIGS. 2 to 7 show examples of attachments in accordance with various embodiments of the invention. FIGS. 2A to 7A show bottom views of various attachments, and FIGS. 2B to 7B show the respective attachments in a side view.

FIGS. 2a and 2b show an attachment 200 which includes a neck portion 116 and a treatment element 118. In this embodiment, the first electrode 120 and the second electrode 122 are formed as metal contacts spaced apart from one another on a surface 128 of the treatment element 118. In the embodiment shown in FIG. 2, the electrodes 120, 122 are round, but electrodes of other shapes are also envisaged. The electrodes 120, 122 in some embodiments extend through the surface 128 of the treatment element 118, such that the electrodes stand out from the surface, as shown in FIG. 2b. In other embodiments, however, the electrodes may be arranged such that they are flush with the surface 128 of the treatment element 118. Arranging the electrodes 120, 122 such that they protrude slightly from the surface 128 of the treatment element 118, may improve the opportunity for the electrodes to make good contact with tissue to be treated.

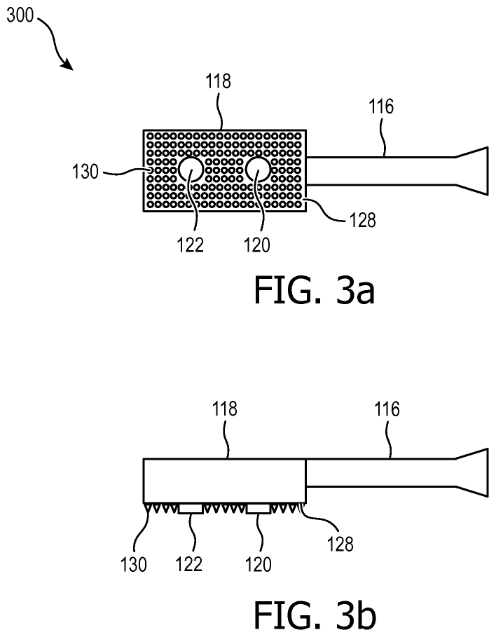

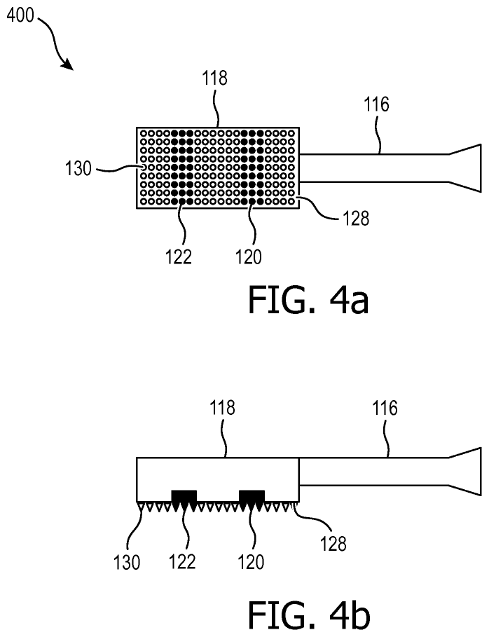

In some embodiments, the first and second electrodes 120, 122 may be incorporated into attachments of oral care devices which are used for other functions, such as cleaning a user's tongue or brushing a user's teeth. In FIGS. 3a, 3b and 4a, 4b, attachments 300 and 400 are shown which function as tongue cleaning attachments. Referring to FIGS. 3a and 3b, the tongue cleaning attachment 300 includes a plurality of tongue-cleaning protrusions 130. The protrusions 130 in this embodiment comprise cones extending from the surface 128 of the treatment element 118, and serve to clean the surface of the tongue as the user moves the attachment of the device around his or her mouth. In other embodiments, the tongue cleaning attachment 300 may include a pattern of ridges or protrusions (not shown) extending from the surface 128, rather than cones 130. In general, the protrusions (e.g. the cones 130, or ridges) serve to penetrate into grooves and indentations in the tongue to provide a thorough cleaning of the various folds and papillae of the tongue. In the embodiment shown in FIG. 3b, the first and second electrodes 120, 122 extend from the surface 128 of the treatment element 118 to approximately the same extent as the cones 130. While the electrodes 120, 122 are shown to be round, disk-like structures in the embodiments of FIG. 3, in other embodiments the electrodes may have other shapes. For example, the electrodes may extend over the length or width of the treatment element 118.

FIGS. 4a and 4b show an example of another arrangement of a tongue cleaning attachment 400 which, like the attachment 300, includes a plurality of protrusions 130 extending from the surface 128 of the treatment element 118. In this embodiment, however, a subset of the cones 130 function as the electrodes 120, 122. The cones that form the electrodes 120, 122 may be formed from a different material than the material used to form the cones that do not function as electrodes. The cones of the first and second electrodes 120, 122 may, in some examples, be formed from a conducting polymer, such as carbon-loaded silicone. In other embodiments, the cones of the first and second electrodes 120, 122 may be formed from one or more other materials, such as a metal or a carbon-based material. As noted above, in alternative embodiments, the protrusions 130 may be ridges or protrusions other than cones. By incorporating electrodes 120, 122 having such shapes, contact between the electrodes and the surface of a user's tongue may be improved, thereby improving the ability of current to flow into the tissue during use.

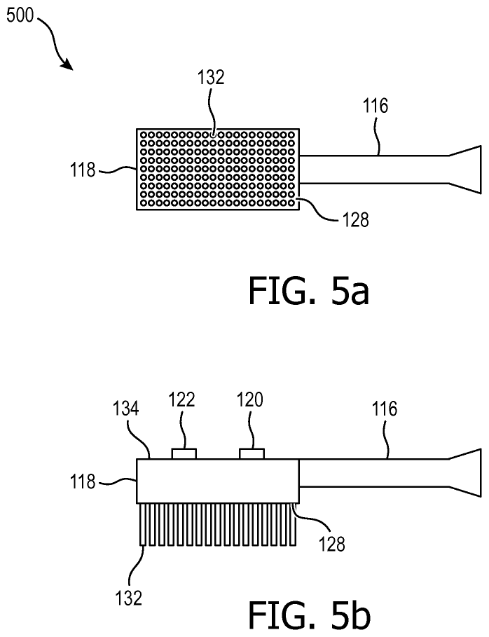

In FIGS. 5a, 5b, 6a, 6b, 7a and 7b, attachments 500, 600 and 700 are shown which function as toothbrush attachments. In some embodiments, each of the attachments 500, 600 and 700 may be for a power toothbrush. When electrically-conductive bristles are implemented in a toothbrush head, an electric current may be conducted from the conductive bristles of the first electrode 120 to the conductive bristles of the second electrode 122 via toothpaste and/or saliva on the bristles and in the user's mouth, rather than via the oral tissue.

Referring to FIGS. 5a and 5b, a power toothbrush attachment, or toothbrush head, 500 is shown. In this embodiment, a plurality of bristles 132 extend from the surface 128 of the treatment element 118. The bristles may be configured to move, for example by vibration and/or rotation, together, in groups or individually, so as to have a cleaning effect on teeth of a user during use. In this embodiment, the first and second electrodes 120, 122 extend from a surface 134 of the treatment element 118. The surface 134 is opposite to the surface 128 from which the bristles 132 extend such that the surface 134 may be considered to be a top or back surface of the toothbrush head 500, and the surface 128 from which the bristles extend may be considered to be a bottom or front surface of the toothbrush head. A user of the attachment 500 of FIG. 5b may use the bristles 132 on the surface 128, for example, to brush his or her teeth, and use the electrodes 120, 122 on the surface 134 to provide electrostimulation to tissue in their mouth. In some embodiments, an electrical current may be delivered to oral tissue via the electrodes 120, 122 while the user is using the toothbrush to brush their teeth. In other embodiments, the user may switch between brushing his or her teeth and using the device for electrostimulation.

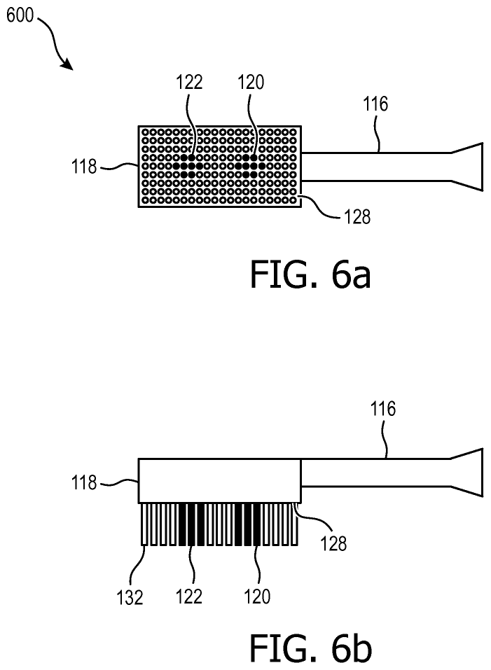

An alternative embodiment is shown in FIGS. 6a and 6b, in which a toothbrush head attachment 600 comprises a plurality of bristles 132 extending from the surface 128 of the treatment element 118. In this embodiment, however, the first electrode 120 comprises a first subset of the plurality of bristles 132 and the second electrode 122 comprises a second subset of the plurality of bristles. Each of the subsets of bristles may include one or more bristles. In the embodiment shown in FIGS. 6a and 6b, the bristles that form the electrodes 120, 122 comprise substantially circular tuft. In other embodiments, however, the bristles forming the electrodes 120, 122 may be arranged in other shapes, such as rows.

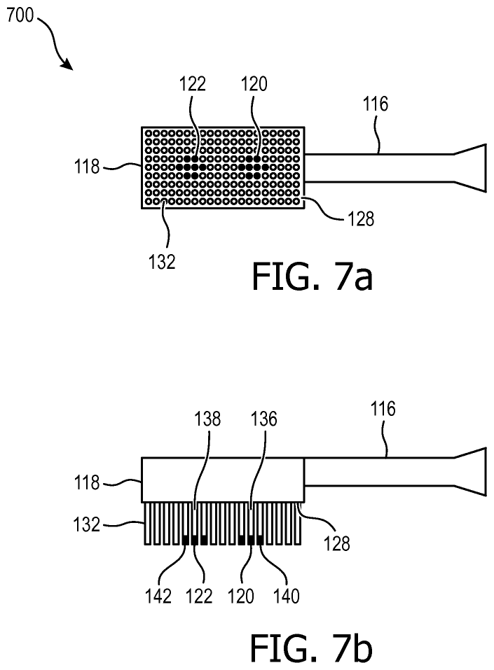

FIGS. 7a and 7b show a toothbrush head attachment 700 which reduces the amount of conduction between the first electrode bristles 120 and the second electrode bristles 122 via routes that don't include the intended oral tissue. As with the attachments 500 and 600, the toothbrush head attachment 700 includes a plurality of bristles 132 extending from the surface 128 of the treatment element 118, and a first subset of the bristles 132 forms the first electrode 120, and a second subset of the bristles 132 forms the second electrode 122. In this embodiment, however, as shown in FIG. 7b, a portion of each of the bristles forming the first electrode 120 includes an electrically-insulated coating 136, and a portion of each of the bristles forming the second electrode 122 include an electrically-insulated coating 138. The first electrode 120 therefore comprises a small portion (in this case the tip) 140 of each bristle in the first subset, and the second electrode 122 comprises a small portion (i.e. the tip) 142 of each bristle in the second subset. In this way, current flowing from one of the electrodes to the other of the electrodes is more likely to be conducted via oral tissue with which the electrodes are in contact, rather than via some other electrically-conducting medium within the oral cavity, such as toothpaste or saliva.

So far various embodiments have been discussed which include only a first electrode 120 and a second electrode 122. In any of the embodiments discussed herein, the attachments may include additional electrodes, for example a third electrode, or third and fourth electrodes (not shown). Electrical currents may be conducted between different combinations of electrodes simultaneously or at different times to one another, such that different portions of tissue can be independently treated with electrostimulation. In some embodiments, different currents may be applied between different electrodes. Similarly, current pulse streams of different patterns may be applied between different electrodes so as to cause a different treatment effect. The current to be applied between electrodes may be controlled by the controller 16 in the oral care device 10.

In embodiments which include more than two electrodes, the electrodes may be arranged in a number of different ways, depending on the intended use of the device 10 or the desired outcome of its use. In some embodiments, the electrodes may be formed as concentric rings (e.g. rings of metal or electrically-conducting cones, protrusions or bristles). In such an example, adjacent rings may have opposite polarities. In other embodiments, electrodes may be arranged at vertices of a polygon, or in a line. The position of the electrodes on the treatment element 118 may be chosen depending on the position of the tissue intended to be treated. For example, electrodes that are intended to deliver a current to tissue at the back of the user's tongue may be positioned at a distal end of the treatment element 118 (i.e. at an end of the treatment element farthest from the body 12 of the oral care device 10).

The electrodes in any of the embodiments described herein may be formed from any suitable electrically-conductive material, such as metal (e.g. copper), carbon, silicone or an electrically-conductive polymer. In some examples, electrodes may be coated with gold.

In addition to an attachment for an oral care device 10, the invention also relates to the oral care device itself. The oral care device 10 includes the handle having a body 12, the drive mechanism 14 within the body of the handle, the drive shaft 20 configured to be driven by the drive mechanism, an attachment 200, 300, 400, 500, 600, 700 as described herein, and a signal generator 11 to generate a first electrical current for delivery to at least one of the first electrode 120 and the second electrode 122. The signal generator 11 may be located in the body 12 of the device 10, or within the attachment. The signal generator 11 may generate a current as a continuous current and/or as a series of pulses, and the current may be applied to one or more additional electrodes independently.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. Any reference signs in the claims should not be construed as limiting the scope.

1. An attachment for an oral care device, the attachment comprising:
a treatment element;
wherein the treatment element comprises a first surface and a second surface opposite to the first surface; and
wherein the treatment element includes a first electrode and a second electrode at least partially embedded within, located on, or extending from one of the first surface and the second surface, the electrodes arranged such that, when the first and second electrodes are placed in contact with tissue within an oral cavity of a user, an applied current is able to flow from the first electrode to the second electrode, via the tissue
wherein the attachment comprises a tongue cleaning attachment or a toothbrush head.

2. An attachment according to claim 1, wherein the first electrode and the second electrode are of opposite polarities.

3. (canceled)

4. An attachment according to claim 1, wherein the oral care device attachment comprises a tongue cleaning attachment.

5. An attachment according to claim 4, wherein the tongue cleaning attachment comprises a plurality of cleaning protrusion;
wherein the first electrode comprises a first subset of the plurality of cleaning protrusions; and
wherein the second electrode comprises a second subset of the plurality of cleaning protrusions.

6. An attachment according to claim 1 wherein at least one of the first electrode and the second electrode comprises at least one of a ridged surface and a surface having a plurality of protrusions.

7. An attachment according to claim 1, wherein the oral care device attachment comprises a toothbrush head.

8. An attachment according to claim 7, wherein the treatment element comprises a first surface and a second surface opposite to the first surface;
wherein a first plurality of bristles extends from the first surface and the first electrode and the second electrode are located on the second surface.

9. An attachment according to claim 7, wherein the toothbrush head comprises a plurality of bristles;
wherein the first electrode comprises a first subset of the plurality of bristles; and
wherein the second electrode comprises a second subset of the plurality of bristles.

10. An attachment according to claim 9, wherein each of the bristles in the first subset and the second subset comprises an insulated portion along a portion of its length.

11. An attachment according to claim 1, wherein the first and second electrodes are formed at least partially of a material selected from a group comprising: gold, copper and an electrically conducting polymer.

12. An oral care device comprising:
a body;
a drive mechanism within the body;
a drive shaft configured to be driven by the drive mechanism;
an attachment according to any of the preceding claims; and
a signal generator to generate a first electrical current for delivery to at least one of the first electrode and the second electrode.

13. An oral care device according to claim 12, wherein the signal generator is located within the attachment.

14. An oral care device according to claim 12, wherein the attachment comprises a third electrode and a fourth electrode; and
wherein the signal generator is configured to generate a second electrical current for delivery to at least one of the third electrode and the fourth electrode, the second electrical current being different from the first electrical current.

15. An oral care device according to claim 12, wherein the signal generator is configured to generate electrical current in a series of pulses.
